(19) United States
(12) Patent Application Publication
(10) Pub. No.: US 2022/0409850 A1
(43) Pub. Date: Dec. 29, 2022

(54) APPARATUS, SYSTEM AND METHOD FOR REDUCING STRESS

(71) Applicant: REFLECT INNOVATION LTD., Herzliya (IL)

(72) Inventor: Noga SAPIR, Tel Aviv (IL)

(21) Appl. No.: 17/771,741

(22) PCT Filed: Oct. 22, 2020
(86) PCT No.: PCT/IL2020/051105

§ 371 Date: Apr. 25, 2022

Related U.S. Application Data
(60) Provisional application No. 62/930,639, filed on Nov. 5, 2019.

(51) Int. Cl. A61M 21/02

(52) U.S. Cl. CPC A61M 21/02; A61M 2021/0022

(57) ABSTRACT

There is provided herein a stress reducing and/or mindfulness inducing apparatus including: a substantially spherically shaped body “huggable” by two hands of a user; at least a first and a second pair of indentations located on an outer surface of said body, wherein each of the first and the second pair of indentations is suitable for placing corresponding index, middle and ring fingers from each hand of the user, wherein each of the first and the second pair of indentations comprises one or more sensors, wherein the one or more sensors are configured to sense one or more parameters associated with the user's stress and/or mindfulness state, wherein the sensors are the same or different from each other; and an indicator having at least two modes of operation, the indicator is configured to provide the user with an indication regarding his/her stress and/or mindfulness state.

### BACKGROUND

Medical research has shown that stress has an effect on health that can be harmful to the body. Stress, by definition, raises a person's blood pressure. Hypertension can increase the risk of a heart attack or stroke. Stress raises heart rate, blood pressure, blood glucose levels, and increased activity of the sympathetic nervous system and may result in a fight-or-flight response which is a physiological change in order to prepare the body for physical activity. Further, relaxed people can make better decisions and learn better.

There are many causes for stress, such as environmental factors such as living under overcrowded and increasingly noisy conditions, experiencing daily events such as coping with traffic, physical activity, undergoing major life changes such as a divorce or death of a loved one, stress in the work place and at home, and finally the use of chemical agents such as alcohol or drugs as a consequence of these stressors.

Stress response can be measured by measuring parameters, such as heart rate, blood pressure, skin conductivity and the like.

There is thus a need for an apparatus, a system and a method for reducing stress and/or inducing mindfulness.

### SUMMARY

Aspects of the disclosure, according to some embodiments thereof, relate to stress reducing and/or mindfulness inducing apparatus, system and method. More specifically, but not exclusively, aspects of the disclosure, according to some embodiments thereof, relate to a stress reducing and/or mindfulness inducing apparatus and system, which include a substantially spherically shaped body “huggable” by two hands of a user, sensors for sensing the user's stress and/or mindfulness state, and an indicator for providing the user with an indication regarding his/her stress and/or mindfulness state.

Thus, according to an aspect of some embodiments, there is provided a stress reducing and/or mindfulness inducing apparatus comprising: a substantially spherically shaped body “huggable” by two hands of a user; at least a first pair and a second pair of indentations located on an outer surface of the body, wherein each of the first and the second pair of indentations comprise two indentations, located on opposing sides of the body surface, and are suitable for placing corresponding fingers from each hand of the user, wherein each of the first and the second pair of indentations comprises one or more sensors, wherein the one or more sensors are configured to sense one or more parameters associated with the user's stress and/or mindfulness state, wherein the sensors are the same or different from each other; and an indicator having at least two modes of operation, a first mode being indicative of a first stress and/or mindfulness state of the user and a second mode being indicative of a second stress and/or mindfulness state of the user.

According to another aspect of some embodiments, there is provided an apparatus as described above, wherein the first pair of indentations comprises one indentation for an index finger of a left hand of the user and another indentation for the index finger of a right hand of the user, and wherein the second pair of indentations comprises one indentation for a middle finger of the left hand of the user and another indentation for the middle finger of the right hand of the user.

According to another aspect of some embodiments, there is provided an apparatus as described above, wherein the first pair of indentations comprises one indentation for any finger of a left hand of the user and another indentation for any finger of a right hand of the user, and wherein the second pair of indentations comprises one indentation for a finger of the left hand of the user and another indentation for the finger of the right hand of the user.

According to another aspect of some embodiments, there is provided an apparatus as described above, wherein the first pair of indentations comprises one indentation for an index, middle or ring finger of a left hand of the user and another indentation for the index, middle or ring finger of a right hand of the user, and wherein the second pair of indentations comprises one indentation for middle or ring finger of the left hand of the user and another indentation for the middle or ring finger of the right hand of the user.

According to another aspect of some embodiments, there is provided an apparatus as described above, wherein the apparatus further comprising a third pair of indentations, wherein the third pair of indentations comprises one indentation for a ring finger of the left hand of the user and another indentation for the ring finger of the right hand of the user.

In some embodiments there is provided a stress reducing and/or mindfulness inducing apparatus comprising: a substantially spherically shaped body “huggable” by two hands of a user; at least a first pair and a second pair of indentations located on an outer surface of the body, wherein each of the first and the second pair of indentations comprise two indentations, located on opposing sides of the body surface, and are suitable for placing corresponding fingers from each hand of the user, wherein the apparatus includes one or more sensors, wherein the one or more sensors are configured to sense one or more parameters associated with the user's stress and/or mindfulness state, wherein the sensors are the same or different from each other; and an indicator having at least two modes of operation, a first mode being indicative of a first stress and/or mindfulness state of the user and a second mode being indicative of a second stress and/or mindfulness state of the user.

According to another aspect of some embodiments, there is provided an apparatus as described above, wherein the apparatus further comprising a processor configured to receive a signal from the one or more sensors, to apply one or more algorithms to the data provided from the signal and to compute an output signal to the indicator.

According to another aspect of some embodiments, there is provided an apparatus as described above, wherein the apparatus is connectable to an external processor configured to receive a signal from the one or more sensors, to apply one or more algorithms to the data provided from the signal and to compute an output signal to the indicator.

According to another aspect of some embodiments, there is provided an apparatus as described above, wherein the one or more algorithms are artificial intelligence based.

According to another aspect of some embodiments, there is provided an apparatus as described above, wherein the apparatus further comprises an energy source.

According to another aspect of some embodiments, there is provided an apparatus as described above, wherein the apparatus further comprises an interface for external charging.

According to another aspect of some embodiments, there is provided an apparatus as described above, wherein the indicator is configured to provide visual, audio, tactile feedback information or any combination thereof.

According to another aspect of some embodiments, there is provided an apparatus as described above, wherein the apparatus, wherein the indicator is a light source. According to another aspect of some embodiments, there is provided an apparatus as described above, wherein the first mode of operation of the light source provide a first color output indicative of a first stress and/or first mindfulness state of the user and the second mode of operation of the light source provides a second color output, different from the first color, indicative of a second stress and/or a second mindfulness state of the user.

According to another aspect of some embodiments, there is provided an apparatus as described above, wherein the first mode of operation of the light source provides light at a first intensity, indicative of a first stress and/or first mindfulness state of the user and a second mode of operation of the light source provides light at a second intensity, different from the first intensity, indicative of a second stress and/or second mindfulness state of the user.

According to another aspect of some embodiments, there is provided an apparatus as described above, wherein the first mode of operation of the light source provides light pulses at a first frequency, indicative of a first stress and/or first mindfulness state of the user and the second mode of operation of the light source provides light pulses at a second frequency, different from the first frequency, indicative of a second stress, and/or second mindfulness state of the user.

According to another aspect of some embodiments, there is provided an apparatus as described above, wherein the indicator is embedded within the spherically shaped body.

According to another aspect of some embodiments, there is provided an apparatus as described above, wherein the indicator is covered by a transparent or semi-transparent cover.

According to another aspect of some embodiments, there is provided an apparatus as described above, wherein the indicator is configured to trigger vibration or expansion of essentially the entire body of the apparatus.

According to another aspect of some embodiments, there is provided an apparatus as described above, wherein the one or more sensors comprise GSR and ECG. According to another aspect of some embodiments, there is provided an apparatus as described above, wherein the one or more sensors comprises a GSR, ECG, PPG, accelerometer, thermometer, barometer, eye tracker, GPS, EMG, pupil size detector, pupil location and/or movement tracker, oximeter, pressure sensor, blood pressure sensor, gyroscope, microphone, camera, fingerprint sensor, humidity sensor, optical sensor, light sensor, photodiode, photoresistor, audiosensor, strain gauge or any combination thereof.

According to another aspect of some embodiments, there is provided an apparatus as described above, wherein the apparatus further comprises communication means functionally connectable to an external device.

According to another aspect of some embodiments, there is provided an apparatus as described above, wherein the communication means comprises wires, cellular communication, Bluetooth, IR, and/or Wi-Fi communication means.

According to another aspect of some embodiments, there is provided an apparatus as described above, wherein the outer surface of the apparatus comprises a soft shell.

According to another aspect of some embodiments, there is provided an apparatus as described above, wherein the soft shell comprises a soft fabric, a foam, a rubber, silicone, flexible plastic, cotton, wool or any combination thereof.

According to another aspect of some embodiments, there is provided an apparatus as described above, wherein the substantially spherically shaped body is in an oval shape or a ball shape.

According to another aspect of some embodiments, there is provided a system for a stress reducing and/or mindfulness inducing apparatus comprising: a substantially spherically shaped body “huggable” by two hands of a user, the substantially spherically shaped body comprises; at least a first pair and a second pair of indentations located on an outer surface of the body, wherein each of the first and the second pair of indentations comprise two indentations, located on opposing sides of the body surface, and are suitable for placing corresponding fingers from each hand of the user, wherein each of the first and the second pair of indentations comprises one or more sensors, wherein the one or more sensors are configured to sense one or more parameters associated with the user's stress and/or mindfulness state, wherein the sensors are the same or different from each other; and an indicator having at least two modes of operation, a first mode being indicative of a first stress and/or mindfulness state of the user and a second mode being indicative of a second stress and/or mindfulness state of the user; and a charger configured to provide power to the a spherically shaped body.

According to another aspect of some embodiments, there is provided a biofeedback method of reducing stress and/or inducing mindfulness comprising: holding the apparatus as described above or the system of as described above using two hands and while placing the fingers in the designated indentations; receiving a first stress and/or mindfulness related indication from the indicator, self-adjusting a stress and/or mindfulness level; and receiving a second stress and/or mindfulness related indication from the indicator.

According to another aspect of some embodiments, there is provided a package that includes the apparatus or the system as described above and a leaflet with instructions regarding how to reach a reduced stress level and/or how to induce mindfulness.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates an isometric view of an exemplary apparatus for stress reducing and/or mindfulness inducing, according to some embodiments;

FIG. 2 illustrates an isometric view of an exemplary system for stress reducing and/or mindfulness inducing, according to some embodiments;

FIG. 3 depicts a user holding a stress reducing and/or mindfulness inducing apparatus with both his/her hands, according to some embodiments; and

FIG. 4 depicts a block diagram of a stress reducing and/or mindfulness inducing system, according to some embodiments.

### DETAILED DESCRIPTION

According to some embodiments, there is provided herein a stress reducing and/or mindfulness inducing apparatus, a system and a biofeedback method for using the apparatus/system for reducing stress and/or inducing mindfulness.

According to some embodiments, the term “inducing mindfulness” may include, for example, but not limited to, inducing meditation, guiding meditation, improving meditation state and/or assisting meditation, inducing focus, controlling/influence/modulate alertness level (e.g., reducing alertness level, increasing alertness level or maintaining essentially constant alertness level), controlling/influence/modulate excitation level, or any combination thereof. Each option presents a separate embodiment.

According to some embodiments, the apparatus has a substantially spherical shape and it is configured to be held (“hugged”) by both hands of the user. The apparatus includes finger indentations. In the indentations, there are sensors (e.g., GSR, ECG or PPG) that measure one or more body parameters, such as heart rate, heart rate variability (HVR), blood pressure, skin conductivity, skin temperature and the like. The indentations are placed such that the hugging of the apparatus is comfortable, i.e. when the fingers are placed in the indentations, the palms of both hands of the user touch (“hug”) the apparatus, which outer surface may comprise a soft shell. The apparatus includes an indicator, such as, but not limited to a display indicator, for example, a LED that produces changing light (changing color/frequency/intensity/pattern or any combination thereof) to provide feedback to the user based on the body's one or more parameter measured and thus assists the user to modulate his/her stress level or mindfulness state, for example, to calm down, to concentrate on a certain task, to control excitation level, to control alertness, to regulate breath, etc.

According to some embodiments, the invention provides a stress reducing and/or mindfulness inducing apparatus comprising: a substantially spherically shaped body “huggable” by two hands of a user; at least a first and a second pair of indentations located on an outer surface of the body, wherein each of the first and the second pair of indentations is suitable for placing corresponding fingers from each hand of the user, wherein each of the first and the second pair of indentations comprises one or more sensors, wherein the one or more sensors are configured to sense one or more parameters associated with the user's stress and/or mindfulness state, wherein the sensors are the same or different from each other; and an indicator having at least two modes of operation, the indicator is configured to provide the user with an indication regarding his/her stress and/or mindfulness state.

In some embodiments, the indicator may provide visual, audible, or tactile information or any combinations thereof. The indicator may be in a form of a light source, such as, in a form of text (wherein the parameter value is given as a number, out of a range of numbers, or as a word) or in some embodiments in a form of a light of different colors, frequencies, intensities, patterns, and the like, that represent the user's stress or mindfulness state. In some embodiments, the indicator is in an audible form and provides sounds in accordance with the user's stress or mindfulness state, such as a sentence, a comment, a word, music, a note, a certain tone, which may be, by a way of example: “you need to relax” or “you did well”, “you are relaxed now”, a certain music as indication of being focused, and the like.

In some embodiments, the indicator includes at least a light source or a sound source.

In some embodiments, the indicator is a lamp that lights up when activated in accordance with the stress or mindfulness state of the user. In some embodiments, the light source is a light emitting diode that lights up when activated in accordance with the stress or mindfulness state of the user.

In some embodiments, the indicator flashes a light when activated in accordance with the stress or mindfulness state.

In some embodiments, a first mode of operation of the indicator provides a first color output indicative of a first stress and/or first mindfulness state of the user, and a second mode of operation of the indicator provides a second color output, different from the first color, indicative of a second stress and/or a second mindfulness state of the user. For example, red may be used for high tension, blue for reduced tension, pink for being calm and green for being relaxed and mindful.

In some embodiments, a first mode of operation of the indicator provides light at a first intensity, indicative of a first stress and/or first mindfulness state of the user, and a second mode of operation of the indicator provides light at a second intensity, different from the first color, indicative of a second stress and/or second mindfulness state of the user.

In some embodiments, a first mode of operation of the indicator provides light pulses at a first frequency, indicative of a first stress and/or first mindfulness state of the user, and a second mode of operation of the indicator provides light pulses at a second frequency, different from the first color, indicative of a second stress and/or second mindfulness state of the user.

In some embodiments, the indicator may be embedded within the spherically shaped body. In some embodiments of the invention, the indicator is covered by a transparent or semi-transparent top.

For example, if the user is stressed, the indicator, which may be a light source, will send a red signal, and once the user relaxes, breathes slowly and/or thinks positive thoughts, the light turns to green. The indicator may have a scale of outputs such as different colors that represents stages of stress or mindfulness.

In some embodiments, the display indicator may be essentially the whole apparatus. In such case, the indication may include vibration or expansion/contraction of essentially the entire body of the apparatus.

In some embodiments, a processor is included in the apparatus or is connected to the apparatus by Wi-Fi or Bluetooth, for example. The processor is configured to receive a signal from the one or more sensors, to apply one or more algorithm to the data provided from the signal, and to compute an output signal to the indicator. In some embodiments the signal results from a combination of at least two parameters (for example, heart rate and skin conductance) and is computed as an output signal to the indicator.

In some embodiments, the apparatus disclosed herein may include an energy source. The energy source may be a battery, single use or rechargeable. In case where the apparatus includes rechargeable battery/batteries, the apparatus further includes an interface for external charging.

In some embodiments, the one or more sensors are capable of measuring skin conductance, respiration rate, heart rate, oxygen saturation, blood pressure, skin moisture and skin temperature. In some embodiments, the one or more sensors include GSR, PPG, ECG or any combination thereof. In some embodiments, the one or more sensors include GSR and ECG.

In some embodiments, the one or more sensors further comprises an accelerometer, thermometer, barometer, eye tracker, GPS, EMG, pupil size detector, pupil location/movement tracker, oximeter, pressure sensor, blood pressure sensor, gyroscope, microphone, camera, fingerprint sensor, humidity sensor, optical sensor, light sensor, photodiode, photoresistor, audiosensor, strain gauge, or any combination thereof.

The term “GSR” (galvanic skin response) refers, according to some embodiments, to a method of or a sensor for measuring the electrical conductance of the skin. Strong emotion can cause stimulus to the sympathetic nervous system that results in more sweat being secreted by the sweat glands. The GSR allows for spotting strong emotions by simply attaching two electrodes to two fingers.

The term “PPG” (photoplethysmography) refers, according to some embodiments, to sensor(s) that use a light-based technology to sense the rate of blood flow as controlled by the heart's pumping action.

The term “ECG sensors” refer, according to some embodiments, to sensors that are capable of delivering heart health metrics and are able to capture more comprehensive signals of overall heart performance, including both Heart Rate (HR) and Heart Rate Variability (HRV).

In some embodiments, the spherically shaped body is in the shape of a ball having a diameter between about 5 cm-30 cm, for example, between about 8 cm-20 cm. The apparatus may be provided in different sizes that may suit the size of the hand of a user.

In some embodiments, the outer surface of the apparatus comprises a soft shell.

In some embodiments, the apparatus is made of a soft fabric such as cotton, linen, wool, or from rubber, a cotton wool, or foam or silicone or any combination thereof.

In some embodiments, the indentations may have a length of between about 2 cm-10 cm and a depth of between about 0.5 mm-20 mm, for example, between about 1 mm-15 mm, 2 mm-13 mm and the like.

In some embodiments, each pair of indentations may have a different size.

In some embodiments, the size (e.g., length and depth) of the indentations may vary from an apparatus to an apparatus in accordance with the size of the apparatus so as to comply with the user's hands.

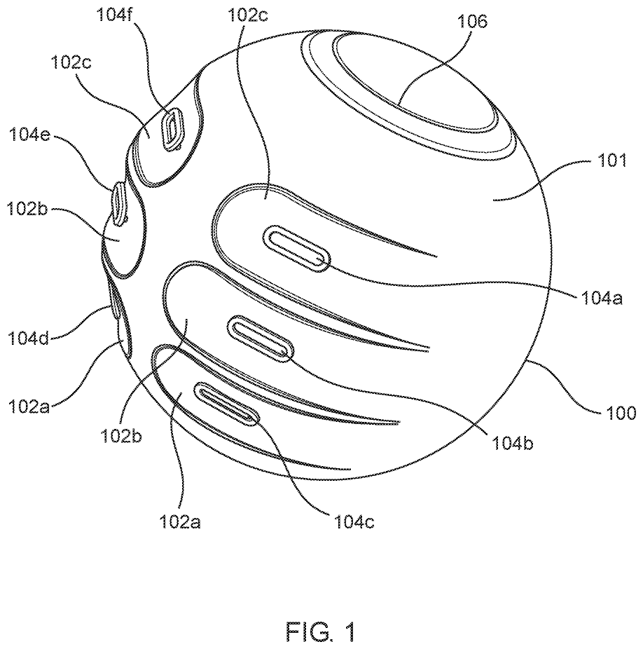

Reference is now made to FIG. 1, which schematically shows, in accordance with some embodiments, an isometric view of an exemplary apparatus 100 for stress reducing and/or mindfulness inducing. Apparatus 100 is shown herein as having ball shaped body 101, though other shapes such as an oval shaped body are also included under the scope of this disclosure. Body 101 has a first 102a, a second 102b and a third 102c pair of indentations located on an outer surface thereof. Each of first, second and third pair of indentations, 102a, 102b and 102c, respectively, is suitable for placing corresponding index, middle and ring fingers of each hand of the user. Each of the first, second and third pair of indentations includes one or more sensors 104a, 104b, 104c, 104d, 104e, 104f, wherein the one or more sensors 104a, 104b, 104c, 104d, 104e, 104f are configured to sense one or more parameters associated with the user's stress and/or mindfulness state. The sensors may be one or more of GSR, PPG, ECG. By a way of example: sensors 104a and 104b are GSR sensors, sensors 104c and 104e are ECG sensors. As another example: sensor 104a may include both GSR sensor and ECG sensor (not shown), such that sensor 104a (GSR) and sensor 104b are both GSR sensors configured to measure skin conductivity, and sensors 104a (ECG), 104f, 104c and 104e (and optionally 104d) are ECG sensors. Body 101 further includes an indicator 106 (e.g., a light source). Display indicator 106 is located on top of apparatus 100 and is functionally connected with one or more of sensors 104a, 104b, 104c, 104d, 104e, 104f. Display indicator 106 has at least two modes of operation; indicator 106 is configured to provide the user with an indication regarding his/her stress and/or mindfulness state, e.g., “very stressed”, “stress relieved”, “relaxed”, “excited”, “focused”, “distracted” etc. Apparatus 100 further includes a socket for a battery and optionally a charging interface (not shown).

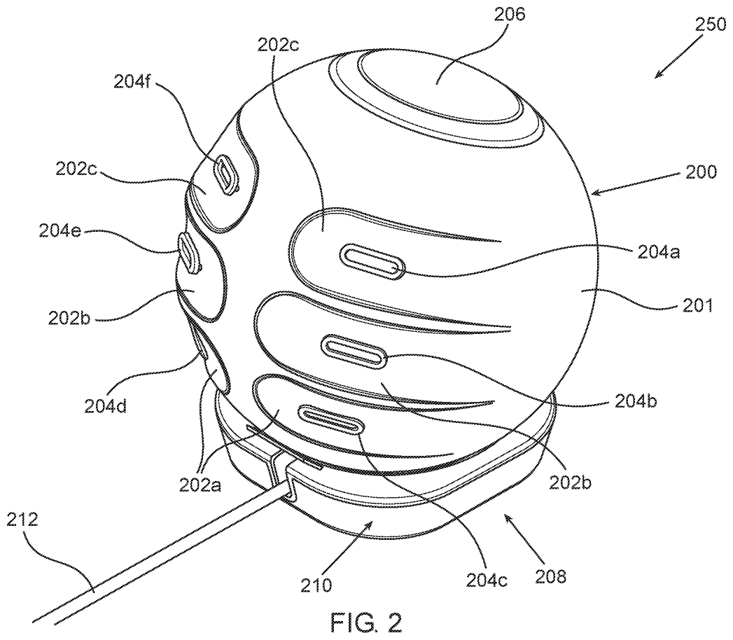

Reference is now made to FIG. 2, which schematically shows, in accordance with some embodiments, an isometric view of an exemplary system 250 including apparatus 200 and an energy source, e.g. a charger 208 having a cable 212.

Apparatus 200 is shown herein as having ball shaped body 201, though other shapes such as an oval shaped body are also included under the scope of this disclosure. Body 201 has a first 102a, a second 202b and a third 202c pair of indentations located on an outer surface thereof. Each of first, second and third pair of indentations, 202a, 202b and 202c, respectively, is suitable for placing corresponding index, middle and ring fingers of each hand of the user. Each of the first, second and third pair of indentations includes one or more sensors 204a, 204b, 204c, 204d, 204e, 204f, wherein the one or more sensors 204a, 204b, 204c, 204d, 204e, 204f are configured to sense one or more parameters associated with the user's stress and/or mindfulness state. The sensors may be one or more of GSR, PPG, ECG. By a way of example: sensors 204a and 204b are PPG, sensors 204c and 204d are GSR and sensors 204e and 204f are ECG. The sensors may be the same or different from each other. Body 201 further includes an indicator 206 (e.g., a display indicator, such as a light source). Indicator 206 is located on top of apparatus 200 and is functionally connected with one or more of sensors 204a, 204b, 204c, 204d, 204e, 204f. Indicator 206 has at least two modes of operation; indicator 206 is configured to provide the user with an indication regarding his/her stress and/or mindfulness state e.g., “very stressed”, “stress relieved”, “relaxed”, “excited”, “focused”, “distracted” etc. Apparatus 200 further includes an energy source, e.g. a charger 208, having a base 210 suitable to the apparatus shape and a cable 212.

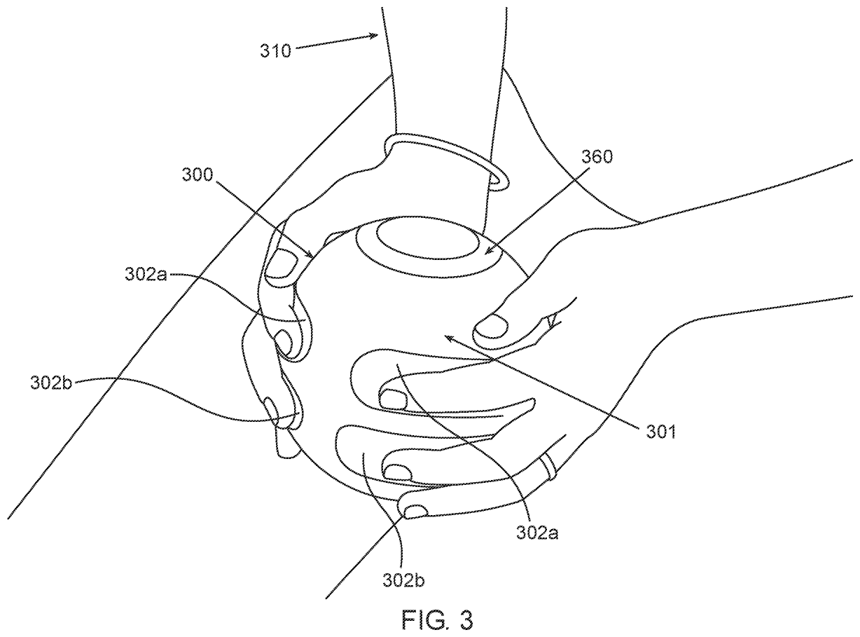

Reference is now made to FIG. 3, which depicts a user 310 holding a stress reducing and/or mindfulness inducing apparatus 300 with both his/her hands. Apparatus 300 is shown herein as having ball shaped body 301 though other shapes such as an oval shaped body are also included under the scope of this disclosure. Body 301 comprises a first 302a, a second 302b and a third (not shown) pair of indentations, respectively, and is suitable for placing corresponding index, middle and ring fingers of each hand of the user. Each of the first, second and third pair of indentations includes one or more sensors (not shown). The sensors may be one or more of GSR, PPG, ECG. The sensors may be the same or different from each other. Body 301 further includes a display indicator 306 (light source). Display indicator 306 is located on top of apparatus 300 and is functionally connected with one or more of sensors (not shown). Display indicator 306 has at least two modes of operation; display indicator 306 is configured to provide the user with an indication regarding his/her stress and/or mindfulness state e.g., “very stressed”, “stress relieved”, “relaxed”, “excited”, “focused”, “distracted” etc. Apparatus 300 further includes an interface for an energy source, e.g., a charger or a battery (not shown).

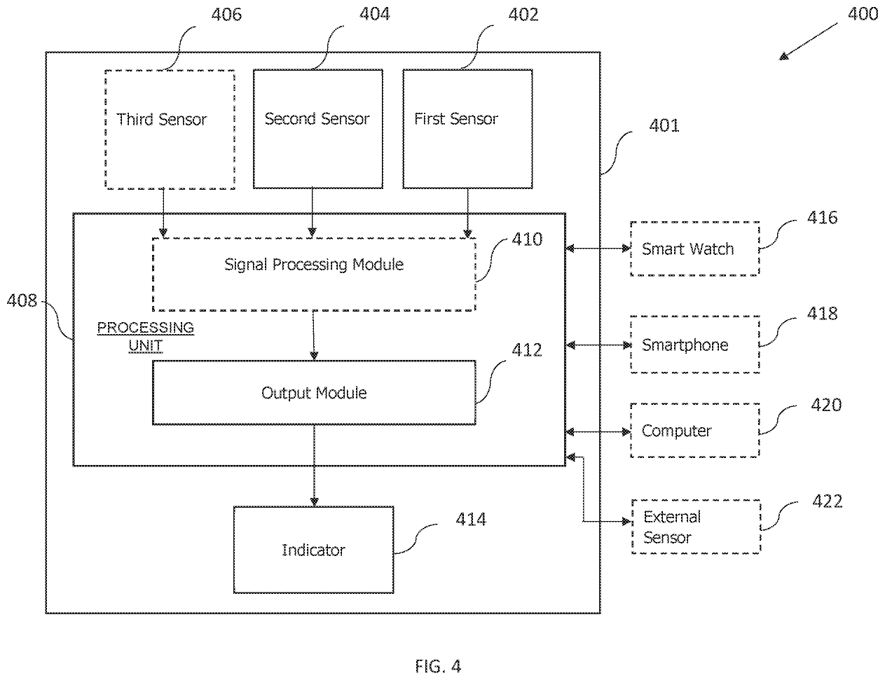

Reference is made now to FIG. 4, which depicts a block diagram of a stress reducing and/or mindfulness inducing system 400, according to some embodiments. System 400 includes a handheld apparatus 401 (which may be similar to apparatus 100, 200 or 300 of FIGS. 1, 2 and 3, respectively), which includes one or more sensors, such as sensors 402, 404 and 406 (and optionally more sensors, not shown), configured to sense one or more parameters associated with the user's stress and/or mindfulness state. Sensors 402, 404 and 406 may be similar, for example, to sensors 104a, 104b, 104c, 104d, 104e, 104f of FIG. 1. System 400 further includes a processing unit 408 which includes a signal processing module 410 and output module 412. Signal processing module 410 is configured to receive signals from the one or more sensors (such as sensors 402, 404 and/or 406), apply one or more algorithms to the data computed from the signals and produce data relating to the user's stress and/or mindfulness state. Output module 412 is configured to provide an output signal indicative of the user's stress and/or mindfulness state, to an indicator 414. According to some embodiments, signal processing module 410 may be external to handheld apparatus 401 (e.g., in and external device such as a mobile connectable device or a wearable device.

Apparatus 401 may communicate with other devices, such as, but not limited to, a smart watch 416, a smartphone 418, a computer 420, and/or a remote server/cloud (not shown) by any type of communication including, but not limited to, Wi-Fi, cellular communication, Bluetooth, IR, wired communication means, etc. Such devices may be utilized, for example, for controlling apparatus 401, operating it, applying certain protocols to it, storing data, adding sensing capabilities, apply changes to algorithms, add display features, provide additional feedback, etc.

System 400 may further includes one or more external sensors, such as an external sensor 422, in an external device, which may be for example, a wearable sensor or a mobile connectable device, a tablet or any computing module adding more sensing capabilities than provided by handheld apparatus 401.

In some embodiments, there is provided a biofeedback method of reducing stress and or inducing mindfulness comprising the step of holding the apparatus as disclosed herein, in accordance with some embodiments, measuring a first stress and/or mindfulness state, relaxing and measuring a second stress and/or mindfulness state.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-26. (canceled)

27. A stress reducing and/or mindfulness inducing apparatus comprising:
a substantially spherically shaped body “huggable” by two hands of a user;
at least a first pair and a second pair of indentations located on an outer surface of said body, wherein each of the first and the second pair of indentations comprise two indentations, located on opposing sides of said body surface, and are suitable for placing corresponding fingers from each hand of the user, wherein each of the first and the second pair of indentations comprises one or more sensors, wherein the one or more sensors are configured to sense one or more parameters associated with the user's stress and/or mindfulness state, wherein the sensors are the same or different from each other; and
an indicator having at least two modes of operation, a first mode being indicative of a first stress and/or mindfulness state of the user and a second mode being indicative of a second stress and/or mindfulness state of the user.

28. The apparatus of claim 27, wherein the first pair of indentations comprises one indentation for an index finger of a left hand of the user and another indentation for the index finger of a right hand of the user, and wherein the second pair of indentations comprises one indentation for a middle finger of the left hand of the user and another indentation for the middle finger of the right hand of the user.

29. The apparatus of claim 28, further comprising a third pair of indentations, wherein the third pair of indentations comprises one indentation for a ring finger of the left hand of the user and another indentation for the ring finger of the right hand of the user.

30. The apparatus of claim 27, further comprising a processor configured to receive a signal from the one or more sensors, to apply one or more algorithms to the data provided from the signal and to compute an output signal to the indicator and/or wherein the apparatus is connectable to an external processor configured to receive a signal from the one or more sensors, to apply one or more algorithms to the data provided from the signal and to compute an output signal to the indicator.

31. The apparatus of claim 27, wherein the one or more algorithms are artificial intelligence based.

32. The apparatus of claim 27, wherein the apparatus further comprises an energy source and/or wherein the apparatus further comprises an interface for external charging.

33. The apparatus of claim 27, wherein the indicator is configured to provide visual, audio, tactile feedback information or any combination thereof.

34. The apparatus of claim 27, wherein the indicator is a light source.

35. The apparatus of claim 34, wherein the first mode of operation of the light source provide a first color output indicative of a first stress and/or first mindfulness state of the user and the second mode of operation of the light source provides a second color output, different from the first color, indicative of a second stress and/or a second mindfulness state of the user.

36. The apparatus of claim 34, wherein the first mode of operation of the light source provides light at a first intensity, indicative of a first stress and/or first mindfulness state of the user and a second mode of operation of the light source provides light at a second intensity, different from the first intensity, indicative of a second stress and/or second mindfulness state of the user.

37. The apparatus of claim 34, wherein the first mode of operation of the light source provides light pulses at a first frequency, indicative of a first stress and/or first mindfulness state of the user and the second mode of operation of the light source provides light pulses at a second frequency, different from the first frequency, indicative of a second stress, and/or second mindfulness state of the user.

38. The apparatus of claim 27, wherein the indicator is embedded within the spherically shaped body and/or wherein the indicator is covered by a transparent or semi-transparent cover.

39. The apparatus of claim 27, wherein the indicator is configured to trigger vibration or expansion of essentially the entire body of the apparatus.

40. The apparatus of claim 27, wherein the one or more sensors comprises a GSR, ECG, PPG, accelerometer, thermometer, barometer, eye tracker, GPS, EMG, pupil size detector, pupil location and/or movement tracker, oximeter, pressure sensor, blood pressure sensor, gyroscope, microphone, camera, fingerprint sensor, humidity sensor, optical sensor, light sensor, photodiode, photoresistor, audiosensor, strain gauge or any combination thereof.

41. The apparatus of claim 27, further comprises communication means functionally connectable to an external device, wherein the communication means comprises wires, cellular communication, Bluetooth, IR, and/or Wi-Fi communication means.

42. The apparatus of claim 27, wherein the outer surface of the apparatus comprises a soft shell comprising a soft fabric, a foam, a rubber, silicone, flexible plastic, cotton, wool or any combination thereof.

43. The apparatus of claim 27, wherein the substantially spherically shaped body is in an oval shape or a ball shape.

44. A system for a stress reducing and/or mindfulness inducing apparatus comprising:
a substantially spherically shaped body “huggable” by two hands of a user, the substantially spherically shaped body comprises;
at least a first pair and a second pair of indentations located on an outer surface of said body, wherein each of the first and the second pair of indentations comprise two indentations, located on opposing sides of said body surface, and are suitable for placing corresponding fingers from each hand of the user, wherein each of the first and the second pair of indentations comprises one or more sensors, wherein the one or more sensors are configured to sense one or more parameters associated with the user's stress and/or mindfulness state, wherein the sensors are the same or different from each other; and
an indicator having at least two modes of operation, a first mode being indicative of a first stress and/or mindfulness state of the user and a second mode being indicative of a second stress and/or mindfulness state of the user; and
a charger configured to provide power to the a spherically shaped body.

45. A biofeedback method of reducing stress and/or inducing mindfulness comprising:
holding the apparatus of claim 1 using two hands and while placing the fingers in the designated indentations; receiving a first stress and/or mindfulness related indication from the indicator, self-adjusting a stress and/or mindfulness level; and receiving a second stress and/or mindfulness related indication from the indicator.

46. A kit comprising the apparatus of claim 1 and a leaflet with instructions regarding how to reach a reduced stress level and/or how to induce mindfulness.
